(19) United States
(12) Patent Application Publication
(10) Pub. No.: US 2002/0082254 A1
(43) Pub. Date: Jun. 27, 2002

(54) Compounds for treating disorders where a decreased level of plasma FFA is desired

(76) Inventors: Hector Beltrandelrio, The Woodlands, TX (US); Poul Jacobsen, Slangerup (DK); Johannes Cornelis De Jong, Bagsvaerd (DK)

Correspondence Address: Reza Green, Esq., Novo Nordisk of North America, Inc., Suite 6400, 405 Lexington Avenue, New York, NY, 10174-6401, US

(21) Appl. No.: 09/931,971

(22) Filed: Aug. 17, 2001

Related U.S. Application Data
(63) Continuation of application No. PCT/DK01/00341, filed on May 15, 2001.
(60) Provisional application No. 60/206,401, filed on May 23, 2000.

(30) Foreign Application Priority Data
May 15, 2000 (DK) PA 2000 00786

(51) Int. Cl. A61K 31/55; A61K 31/445; A61K 31/40; C07D 211/56; C07D 207/10; C07D 223/12

(52) U.S. Cl. 514/217.11; 514/329; 514/426; 546/223; 548/557; 540/606

(57) ABSTRACT

The present invention relates to novel compounds, compositions containing them, and their use for treating medical disorders where a decreased level of plasma free fatty acids (FFA) is desired.

### FIELD OF INVENTION

The present invention relates to novel compounds, compositions containing them, and their use for treating medical disorders where a decreased level of plasma free fatty acids (FFA) is desired.

### BACKGROUND OF THE INVENTION

The overall energy homeostasis of a mammalian system requires a high degree of regulation to ensure the availability of the appropriate substrate at the appropriate time. Plasma glucose levels rise during the post-prandial state, to return to pre-prandial levels within 2-3 hours. During these 2-3 hours, insulin promotes glucose uptake by skeletal muscle and adipose tissue and decreases the release of free fatty acids (FFA) from adipocytes, to ensure that the two substrates do not compete with each other. When plasma glucose levels fall, an elevation in plasma FFA is necessary to switch from glucose to fat utilization by the various tissues.

In individuals with insulin resistance, FFA levels do not fall in response to insulin, as they do in normal individuals, preventing the normal utilization of glucose by skeletal muscle, adipose and liver. Furthermore, there is a negative correlation between insulin sensitivity and plasma FFA levels.

Hormone-sensitive lipase (HSL) is an enzyme, expressed primarily in adipocytes, that catalyses the conversion of triglycerides to glycerol and fatty acids. It is through the regulation of this enzyme that the levels of circulating FFA are modulated. Insulin leads to the inactivation of HSL with a subsequent fall in plasma FFA levels during the post-prandial state, followed by the activation of the enzyme when the insulin concentration falls and catecholamines rise during the post-absorptive period. The activation of HSL leads to an increase in plasma FFA, as they become the main source of energy during fasting.

The activation-inactivation of HSL is mediated through the cAMP-protein kinase A and AMP-dependent kinase pathways. There are compounds, such as nicotinic acid and its derivatives, that decrease the activation of HSL via these pathways and cause a decrease in lipolysis that leads to a reduction in the FFA levels. These drugs have a beneficial effect in the utilization of glucose and in the normalization of the excess triglyceride synthesis seen in patients with elevated FFA. However, since these pathways are used by other processes in the body, these drugs have severe side effects.

We have now found compounds that specifically inhibit the lipolytic activity of HSL and lead to a decrease in plasma FFA levels. These compounds can be used to treat disorders where a decreased level of plasma FFA is desired, such as insulin resistance, dyslipidemia and abnormalities of lipoprotein metabolism.

One object of the present invention is thus to provide compounds that specifically inhibit the lipolytic activity of HSL. A further object is to provide compounds which have good oral bioavailability.

### SUMMARY OF THE INVENTION

One aspect of the present invention thus provides novel compounds that specifically inhibit the lipolytic activity of HSL.

These compounds can be utilized in vitro as unique research tools for understanding, inter alia, how the lipolytic activity of HSL is regulated at the adipocyte level.

Moreover, the compounds can also be administered in vivo for inhibiting the lipolytic activity of HSL.

### DESCRIPTION OF THE INVENTION

Accordingly, the present invention relates to a compound of general formula I

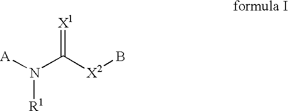

wherein

A is a nitrogen containing ring system attached through the nitrogen atom, which nitrogen containing ring system is optionally substituted with one or more substituents independently selected from halogen, hydroxy, amino, oxy, cyano, nitro, C₁₋₆-alkyl and C₁₋₆-alkoxy;

wherein each of the C₁₋₆-alkyl or C₁₋₆-alkoxy may optionally be substituted with one or more substituents independently selected from hydroxy, halogen, amino, cyano and nitro;

R¹ is hydrogen or C₁₋₆-alkyl;

X¹ is —S— or —O—;

X² is —S— or —O—;

B is aryl optionally substituted with one or more substituents independently selected from halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl and —C₁₋₆-alkyl-NR²⁰—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁰ is hydrogen or C₁₋₆-alkyl;

or B is

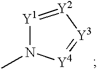

wherein

Y¹ is —C(R²¹)═ or —N═;

Y² is —C(R²²)═ or —N═;

Y³ is —C(R²³)═ or —N═;

Y⁴ is —C(R²⁴)═ or —N═;

wherein R²¹, R²², R²³ and R²⁴ independently are hydrogen, halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁵—C(═O)—C₁₋₆-alkyl, —NR²⁵—C(═O)—C₁₋₆-alkoxy, —NR²⁵—C₁₋₆-alkyl or —C₁₋₆-alkyl-NR²⁵—C₁₋₆-alkyl;

wherein each of the C₁₋₆-alkyl, C₁₋₆-alkoxy, C₃₋₁₀-cycloalkyl, or aryl may optionally be substituted with one or more substituents independently selected from hydroxy, halogen, amino, cyano and nitro;

R²⁵ is hydrogen or C₁₋₆-alkyl;

or a pharmaceutically acceptable salt thereof.

Moreover, the compounds of formula I may comprise any optical isomers thereof, in the form of separated, pure or partially purified optical isomers or racemic mixtures thereof.

Whenever one or more chiral carbon atoms are present, such chiral center or centers may be in the R- or S-configuration, or a mixture of R and S.

In one embodiment of the compound of formula I, A is

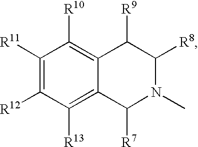

wherein

R⁷, R⁸, and R⁹ independently are hydrogen or C₁₋₆-alkyl;

R¹⁰, R¹¹, R¹², and R¹³ independently are hydrogen, halogen, C₁₋₆-alkyl or C₁₋₆-alkoxy.

In one embodiment R⁷ is hydrogen. In a second embodiment R⁷ is C₁₋₆-alkyl, in particular methyl. In a third embodiment R⁸ is hydrogen. In a further embodiment R⁸ is C₁₋₆-alkyl, in particular methyl. In a still further embodiment R⁹ is hydrogen. In a further embodiment R¹⁰ is hydrogen. In a still further embodiment R¹¹ is hydrogen. In a further embodiment R¹¹ is C₁₋₆-alkoxy, in particular methoxy. In a still further embodiment R¹² is hydrogen. In a further embodiment R¹² is C₁₋₆-alkoxy, in particular methoxy. In a still further embodiment R¹³ is hydrogen.

In a further embodiment of the compound of formula I, A is

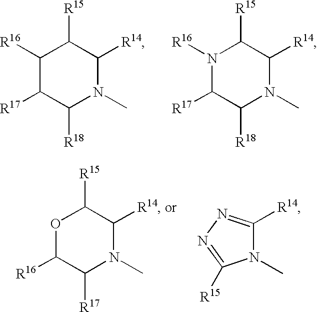

wherein R¹⁴, R¹⁵, R¹⁶, R¹⁷ and R¹⁸ independently are hydrogen, halogen, C₁₋₆-alkyl or C₁₋₆-alkoxy.

In one embodiment A is

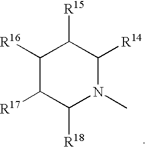

In a second embodiment A is

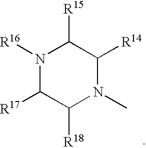

In a third embodiment A is

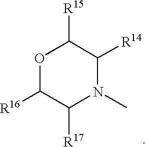

In a further embodiment A is

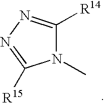

In a still further embodiment R¹⁴ is hydrogen. In a further embodiment R¹⁵ is hydrogen. In a still further embodiment R¹⁶ is hydrogen. In a further embodiment R¹⁷ is hydrogen. In a still further embodiment R¹⁸ is hydrogen.

In a still further embodiment of the compound of formula I, in the nitrogen containing ring system A at least one of the atoms next to the nitrogen atom through which A is attached is unsubstituted.

In the compound of the above formula I, A is preferably 3,4-dihydro-1H-isoquinolin-2-yl, 6,7-dimethoxy-3,4-dihydro-1H-isoquinolin-2-yl, 1-methyl-3,4-dihydro-1H-isoquinolin-2-yl, 3-methyl-3,4-dihydro-1H-isoquinolin-2-yl, piperidin-1-yl or morpholin-4-yl.

In a still further embodiment of the compound of formula I, R¹ is hydrogen. In a further embodiment R¹ is C₁₋₆-alkyl, in particular methyl.

In a still further embodiment of the compound of formula I, X¹ is —S—. In a further embodiment X¹ is —O—.

In a still further embodiment of the compound of formula I, X² is —S—. In a further embodiment X² is —O—.

In a still further embodiment of the compound of formula I, X¹ is —O— and X² is —O—. In a further embodiment of the compound of formula I, X¹ is —S— and X² is —S—.

In a still further embodiment of the compound of formula I, B is aryl optionally substituted with one or more substituents independently selected from

halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl and —C₁₋₆-alkyl-NR²⁰—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁰ is hydrogen or C₁₋₆-alkyl.

In one embodiment, B is aryl. In a second embodiment, B is aryl substituted with one or more halogen, in particular Br, F or Cl. In a third embodiment, B is aryl substituted with amino. In a further embodiment, B is aryl substituted with C₁₋₆-alkyl, such as tert-butyl, butyl-2-yl, 2-methyl-butyl-2-yl, propyl, ethyl or methyl. In a still further embodiment, B is aryl substituted with C₁₋₆-alkoxy, such as pentoxy, butoxy, propoxy or methoxy, In a further embodiment, B is aryl substituted with C₁₋₆-alkylcarbonyl, such as ethylcarbonyl or methylcarbonyl. In a still further embodiment, B is aryl substituted with C₁₋₆-alkoxycarbonyl, such as ethoxycarbonyl or methoxycarbonyl. In a further embodiment, B is aryl substituted with C₃₋₁₀-cycloalkyl, such as cyclohexyl. In a still further embodiment, B is aryl substituted with aryl, such as phenyl. In a further embodiment, B is aryl substituted with aryl-C₁₋₆-alkyl, such as phenylmethyl. In a still further embodiment, B is aryl substituted with aryl-C₁₋₆-alkoxy, such as phenylmethoxy. In a further embodiment, B is aryl substituted with aryl-C₁₋₆-alkoxycarbonyl, such as phenylmethoxycarbonyl. In a still further embodiment, B is aryl substituted with arylcarbonyl, such as 4-methyl-phenylcarbonyl. In a further embodiment, B is aryl substituted with —NR²⁰—C(═O)—C₁₋₆-alkyl, such as methylcarbonylamino. In a still further embodiment, B is aryl substituted with —NR²⁰—C(═O)—C₁₋₆-alkoxy, such as tert-butoxycarbonylamino. In a further embodiment, B is aryl substituted with —NR²⁰—C₁₋₆-alkyl. In a still further embodiment, B is aryl substituted with —C₁₋₆-alkyl-NR²⁰—C₁₋₆-alkyl, such as dimethylaminoethyl.

In a still further embodiment of the compound of formula I, B is

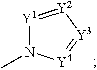

wherein

Y¹ is —C(R²¹)═ or —N═;

Y² is —C(R²²)═ or —N═;

Y³ is —C(R²³)═ or —N═;

Y⁴ is —C(R²⁴)═ or —N═;

wherein R²¹, R²², R²³, and R²⁴ independently are hydrogen, halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl-C₁₋₆-alkyl, aryl-C₁₋₆-alkoxy, aryl-C₁₋₆-alkoxycarbonyl, arylcarbonyl, —NR²⁵—C(═O)—C₁₋₆-alkyl, —NR²⁵—C(═O)—C₁₋₆-alkoxy, —NR²⁵—C₁₋₆-alkyl or —C₁₋₆-alkyl-NR²⁵—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁵ is hydrogen or C₁₋₆-alkyl.

In one embodiment, yt is —CH═. In a second embodiment, Y² is —N═. In a third embodiment, Y³ is —CH═. In a further embodiment Y⁴ is —N═.

In a still further embodiment of the compound of formula I, B is

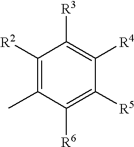

wherein

R², R³, R⁴, R⁵ and R⁶ independently of each other are hydrogen, halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl or —C₁₋₆-alkyl-NR²⁰—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁰ is hydrogen or C₁₋₆-alkyl.

In a still further embodiment of the compound of formula I, B is

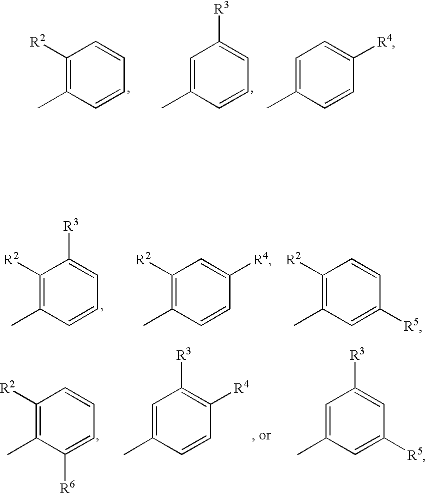

wherein R², R³, R⁴, R⁵ and R⁶ are defined as above. In one embodiment, B is

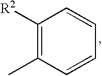

such as 2-methyl-phenyl, 2-methoxy-phenyl, 2-bromo-phenyl, 2-fluoro-phenyl. In a second embodiment, B is

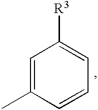

such as 3-methyl-phenyl. In a further embodiment, B is

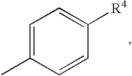

such as 4-methoxy-phenyl, 4-methoxy-carbonyl-phenyl, 4-bromo-phenyl, 4-chloro-phenyl, 4-fluoro-phenyl, 4-methyl-phenyl, 4-trifluoromethyl-phenyl, 4-tert-butoxycarbonylamino-phenyl, 4-amino-phenyl, 4-phenyl-ethoxycarbonyl-phenyl, 4-propyl-phenyl, 4-methoxy-amino-phenyl, 4-(4-methyl-carbonyl)-phenyl, 4-phenyl-methyl-phenyl, 4-ethyl-phenyl, 4-phenyl-methoxy-phenyl, 4-butoxy-phenyl, 4-(2-methyl-propyl-2-yl), 4-(propyl-2-yl), 4-tert-butyl-phenyl, 4-dimethyl-amino-ethyl-phenyl, 4-ethoxy-carbonyl-phenyl, 4-bi-phenyl, 4-pentoxy-phenyl, 4-phenoxy-phenyl, 4-propoxy-phenyl, 4-triflouromethoxy-phenyl, 4-phenyl-carbonyl-phenyl, 4-methoxy-carbonyl-methyl-phenyl, 4-cyclohexyl-phenyl, 4-chloro-propyl-carbonyl-phenyl. In a still further embodiment, B is

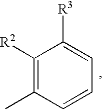

In a further embodiment, B is

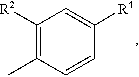

such as 2,4-dichloro-phenyl. In a still further embodiment, B is

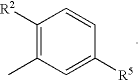

In a further embodiment B is

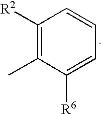

In a still further embodiment, B is

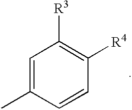

In a further embodiment, B is

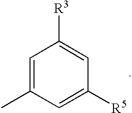

In a still further embodiment of the compound of formula I, B is

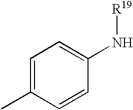

wherein R¹⁹ is hydrogen, C₁₋₆-alkyl, C₁₋₆-alkylcarbonyl, C₁₋₆-alkoxycarbonyl, aryl-C₁₋₆-alkyl, aryl-C₁₋₆-alkoxycarbonyl or arylcarbonyl. In one embodiment, B is 4-amino-phenyl. In a second embodiment, B is 4-methyl-carbonyl-amino-phenyl.

Any possible combination of two or more of the embodiments described herein is comprised within the scope of the invention.

Preferred compounds of formula I of the invention are:

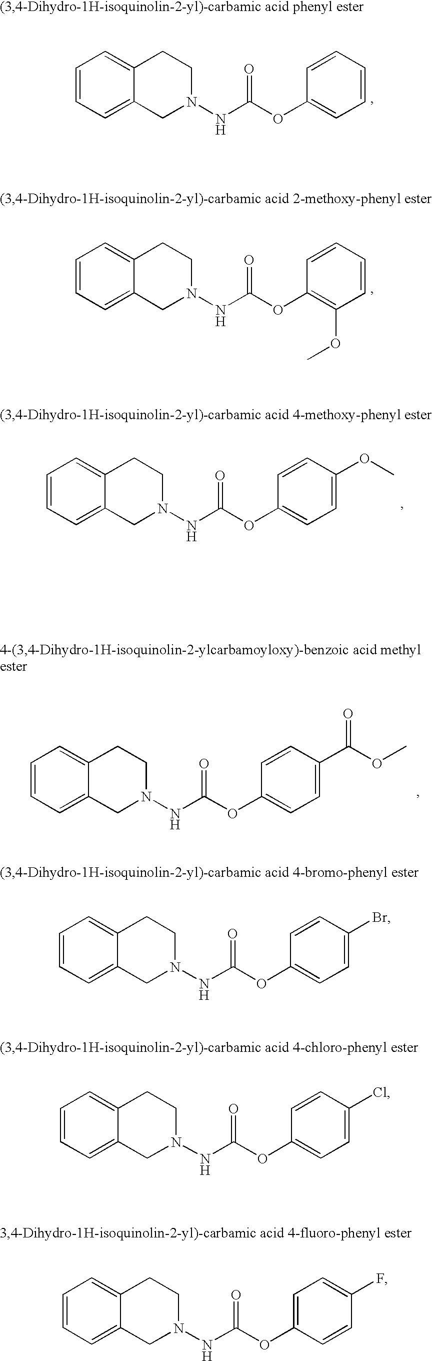

and pharmaceutically acceptable salts thereof.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates which the present compounds are able to form.

Any possible combination of two or more of the embodiments described herein is comprised within the scope of the present invention.

Pharmaceutical Composition

In a still further aspect, the invention relates to a pharmaceutical composition comprising, as an active ingredient, a compound of general formula I

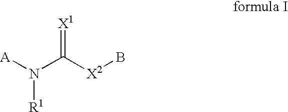

wherein

A is a nitrogen containing ring system attached through the nitrogen atom, which nitrogen containing ring system is optionally substituted with one or more substituents independently selected from halogen, hydroxy, amino, oxy, cyano, nitro, C₁₋₆-alkyl and C₁₋₆-alkoxy;

wherein each of the C₁₋₆-alkyl, or C₁₋₆-alkoxy may optionally be substituted with one or more substituents independently selected from hydroxy, halogen, amino, cyano and nitro;

R¹ is hydrogen or C₁₋₆-alkyl;

X¹ is —S— or —O—;

X² is —S— or —O—;

B is aryl optionally substituted with one or more substituents independently selected from halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl and —C₁₋₆-alkyl—NR²⁰—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁰ is hydrogen or C₁₋₆-alkyl;

or B is

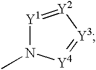

wherein

Y¹ is —C(R²¹)═ or —N═;

Y² is —C(R²²)═ or —N═;

Y³ is —C(R²³)═ or —N═;

Y⁴ is —C(R²⁴)═ or —N═;

wherein R²¹, R²², R²³ and R²⁴ independently are hydrogen, halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁵—C(═O)—C₁₋₆-alkyl, —NR²⁵—C(═O)—C₁₋₆-alkoxy, —NR²⁵—C₁₋₆-alkyl or —C₁₋₆-alkyl-NR²⁵—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁵ is hydrogen or C₁₋₆-alkyl; or a pharmaceutically acceptable salt thereof together with a pharmaceutically acceptable carrier.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants, etc.

The therapeutic dose of the compound will depend upon the frequency and mode of administration, the sex, age, weight and general condition of the subject treated, the nature and severity of the condition treated and any concomitant diseases to be treated, and other factors evident to those skilled in the art. The formulations may conveniently be presented in unit dosage form by methods known to those skilled in the art. In one embodiment, the composition in unit dosage form comprises from about 0.05 to about 2000 mg, preferably from about 0.1 to about 500 mg of the compound of formula I or a pharmaceutically acceptable salt thereof.

In a still further embodiment, the pharmaceutical composition is for oral, nasal, transdermal, pulmonary or parenteral administration.

For parenteral routes, such as intravenous, intrathecal, intramuscular and similar administration, typical doses are of the order of about half the dose employed for oral administration.

The compounds of this invention are generally utilized as the free substance or as a pharmaceutically acceptable salt thereof. One example is an acid addition salt of a compound having the a free base functionality. When a compound of the invention contains a free base functionality, such salts are prepared in a conventional manner by treating a solution or suspension of the free base form of the compound with a chemical equivalent of a pharmaceutically acceptable acid, for example an inorganic or organic acid. Representative examples hereof are mentioned above. Physiologically acceptable salts of a compound with a hydroxy group include the anion of said compound in combination with a suitable cation, such as sodium or ammonium ion.

For parenteral administration, solutions of the present compounds in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the compounds of the invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain:

| | |
|---|---|
| Core: | |
| Active compound (as free compound or salt thereof) | 5 mg |
| Colloidal silicon dioxide (Aerosil ™) | 1.5 mg |
| Cellulose, microcryst. (Avicel ™) | 70 mg |
| Modified cellulose gum (Ac-Di-Sol ™) | 7.5 mg |
| Magnesium stearate | q.s. |
| Coating: | |
| Hydroxypropylmethylcellulose (HPMC) approx. | 9 mg |
| *Mywacett ™ 9-40 T approx. | 0.9 mg |

*Acylated monoglyceride used as plasticizer for film coating.

The compounds of the invention may be administered to a mammal, especially a human, in need thereof. Such mammals include also animals, both domestic animals, e.g. household pets, and non-domestic animals such as wildlife.

In a further aspect of the invention the present compounds may be administered in combination with further pharmacologically active substances e.g. an antidiabetic or other pharmacologically active material, including other compounds for the treatment and/or prevention of insulin resistance and diseases, wherein insulin resistance is the pathophysiological mechanism.

Furthermore, the compounds according to the invention may be administered in combination with antiobesity agents or appetite regulating agents.

In a further embodiment the invention relates to a pharmaceutical composition for specifically inhibiting the lipolytic activity of HSL, the composition comprising, as an active ingredient, a compound of formula I or a pharmaceutically acceptable salt thereof together with a pharmaceutically acceptable carrier or diluent.

It has been demonstrated that compounds of the general formula I possess the ability to specifically inhibit HSL in vivo. The compounds may therefore be used in the treatment of conditions which require decreased plasma FFA.

In a still further aspect, the present invention relates to the use of of a compound that specifically inhibits the lipolytic activity of HSL, or a pharmaceutically acceptable salt thereof, for the preparation of a medicament for the treatment of a disorder where a decreased level of plasma FFA is desired.

In one embodiment, the disorder where a decreased level of plasma FFA is desired is diabetes type 2, insulin resistance, impaired glucose tolerance, hyperglycemia, dyslipidemia, or abnormalities of lipoprotein metabolism.

In a further embodiment, the compound that specifically inhibits lipolytic activity of HSL is a compound of general formula I

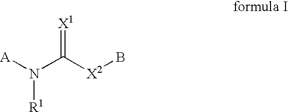

wherein

A is a nitrogen containing ring system attached through the nitrogen atom, which nitrogen containing ring system is optionally substituted with one or more substituents independently selected from halogen, hydroxy, amino, oxy, cyano, nitro, C₁₋₆-alkyl and C₁₋₆-alkoxy;

wherein each of the C₁₋₆-alkyl or C₁₋₆-alkoxy may optionally be substituted with one or more substituents independently selected from hydroxy, halogen, amino, cyano and nitro;

R¹ is hydrogen or C₁₋₆-alkyl;

X¹ is —S— or —O—;

X² is —S— or —O—;

B is aryl optionally substituted with one or more substituents independently selected from halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl and —C₁₋₆alkyl-NR²⁰—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁰ is hydrogen or C₁₋₆-alkyl;

or B is

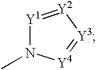

wherein

Y¹ is —C(R²¹)═ or —N═;

Y² is —C(R²²)═ or —N═;

Y³ is —C(R²³)═ or —N═;

Y⁴ is —C(R²⁴)═ or —N═;

wherein R²¹, R²², R²³ and R²⁴ independently are hydrogen, halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆alkyl, aryl-C₁₋₆-alkoxy, aryl-C₁₋₆-alkoxycarbonyl, arylcarbonyl, —NR²⁵—C(═O)—C₁₋₆-alkyl, —NR²⁵—C(═O)—C₁₋₆-alkoxy, —NR²⁵—C₁₋₆-alkyl or —C₁₋₆-alkyl-NR²⁵—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁵ is hydrogen or C₁₋₆-alkyl;

or a pharmaceutically acceptable salt thereof, together with a pharmaceutically acceptable carrier.

In a still further aspect, the invention relates to a method of treating disorders of a mammal, e.g. a human, where a decreased level of plasma FFA is desired, the method comprising administering to said mammal an effective amount of a compound that specifically inhibits the lipolytic activity of HSL, or a pharmaceutically acceptable salt thereof.

In one embodiment of the method, the disorder where a decreased level of plasma FFA is desired is diabetes type 2, insulin resistance, impaired glucose tolerance, hyperglycemia, dyslipidemia, or abnormalities of lipoprotein metabolism.

In a further embodiment, the compound that specifically inhibits lipolytic activity of HSL is a compound of general formula I

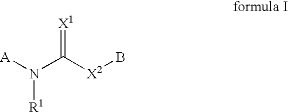

wherein

A is a nitrogen containing ring system attached through the nitrogen atom, which nitrogen containing ring system is optionally substituted with one or more substituents independently selected from halogen, hydroxy, amino, oxy, cyano, nitro, C₁₋₆-alkyl and C₁₋₆-alkoxy;

wherein each of the C₁₋₆-alkyl or C₁₋₆-alkoxy may optionally be substituted with one or more substituents independently selected from hydroxy, halogen, amino, cyano and nitro;

R¹ is hydrogen or C₁₋₆-alkyl;

X¹ is —S— or —O—;

X² is —S— or —O—;

B is aryl optionally substituted with one or more substituents independently selected from halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl and —C₁₋₆-alkyl-NR²⁰—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁰ is hydrogen or C₁₋₆-alkyl;

or B is

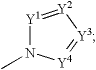

wherein

Y¹ is —C(R²¹)═ or —N═;

Y² is —C(R²²)═ or —N═;

Y³ is —C(R²³)═ or —N═;

Y⁴ is —C(R²⁴)═ or —N═;

wherein R²¹, R²², R²³ and R²⁴ independently are hydrogen, halogen, hydroxy, amino, cyano, nitro, C₁₋₆alkyl, C₁₋₆-alkoxy, C₁₋₆-alkylcarbonyl, C₁₋₆-alkoxycarbonyl, C₃₋₁₀-cycloalkyl, aryl, aryl-C₁₋₆alkyl, aryl-C₁₋₆-alkoxy, aryl-C₁₋₆-alkoxycarbonyl, arylcarbonyl, —NR²⁵—C(═O)—C₁₋₆-alkyl, —NR²⁵—C(═O)—C₁₋₆-alkoxy, —NR²⁵—C₁₋₆-alkyl or —C₁₋₆-alkyl-NR²⁵—C₁₋₆-alkyl;

wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;

R²⁵ is hydrogen or C₁₋₆-alkyl;

or a pharmaceutically acceptable salt thereof, together with a pharmaceutically acceptable carrier.

In a still further embodiment, the administration is carried out by the oral, nasal, transdermal, pulmonary or parenteral route.

In a still further aspect, the present invention relates to a method of identifying compounds for the treatment of disorders where a decreased level of plasma FFA is desired, characterised by screening out compounds that specifically inhibit HSL.

The specific inhibition of HSL can, e.g., be determined by the use of the assays as described in the assays under “Pharmacological methods” below.

In one embodiment of the invention, the compounds that specifically inhibit HSL give rise to at most about 20 percent inhibition, such as less than 20 percent inhibition, of the lipases lipoprotein lipase (LPL) and hepatic lipase (HL) at a concentration of 50 μM. In a further embodiment, the compounds that specifically inhibit HSL give rise to less than 10 percent inhibition of the lipases LPL and HL at a concentration of 50 μM.

In a still further embodiment, the compounds that specifically inhibit HSL have an inhibition constant (with respect to inhibition of HSL), Kᵢ, of less than 10 μM. In a further embodiment, the compounds that specifically inhibit HSL have a Kᵢ (with respect to HSL inhibition) of less than 1 μM. Kᵢ is defined in the normal manner, i.e.

Kᵢ=([enzyme][inhibitor])/[enzyme-inhibitor complex]

where the terms in square brackets are equilibrium concentrations of the species in question.

Within the context of the present invention, treatment is to be understood as treatment and/or prevention.

The present invention is further illustrated by the following examples; these examples are, however, not to be construed as limiting the scope of protection.

Definitions

The following provides detailed definitions of the terms used to describe the compounds of the invention:

“Halogen” designates an atom selected from the group consisting of F, Cl, Br and I.

The term “C₁₋₆-alkyl” in the present context designates a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆-alkoxy” in the present context designates a group —O—C₁₋₆-alkyl wherein C₁₋₆-alkyl is as defined above. Representative examples include, but are not limited to, methoxy, ethoxy, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₃₋₁₀-cycloalkyl” as used herein represents a saturated mono-, bi-, tri- or spiro-carbocyclic group having from 3 to 10 carbon atoms. Representative examples are cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₈-heterocyclyl” as used herein represents a saturated 3 to 8 membered ring containing one or more heteroatoms selected from nitrogen, oxygen and sulfur. Representative examples are pyrrolidyl, piperidyl, piperazinyl, morpholinyl, thiomorpholinyl, aziridinyl, tetrahydrofuranyl and the like.

The term “aryl” as used herein represents a carbocyclic aromatic ring system being either monocyclic, bicyclic, or polycyclic, such as phenyl, biphenyl, naphthyl, anthracenyl, phenanthrenyl, fluorenyl, indenyl, pentalenyl, azulenyl, biphenylenyl and the like. Aryl is also intended to include the partially hydrogenated derivatives of the carbocyclic aromatic systems enumerated above. Non-limiting examples of such partially hydrogenated derivatives are 1,2,3,4-tetrahydronaphthyl, 1,4-dihydronaphthyl and the like.

The term “heteroaryl” as used herein represents a heterocyclic aromatic ring system containing one or more heteroatoms selected from nitrogen, oxygen and sulfur such as furanyl, thiophenyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, 3,4-dihydroisoquinolinyl, pyrrolinyl, pyrazolinyl, indolinyl, oxazolidinyl, oxazolinyl, oxazepinyl and the like.

The term “heterocyclic system” as used herein includes aromatic as well as non-aromatic ring moieties, which may be monocyclic, bicyclic or polycyclic, and containing in their ring structure one or more heteroatoms selected from nitrogen, oxygen and sulfur. Non-limiting examples of such heterocyclic systems are C₃₋₈-heterocyclyl and heteroaryl.

The term “nitrogen containing ring system” designates a heterocyclic system as defined herein having at least one nitrogen atom.

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent, the substituents may be the same or different.

Pharmacological Methods

Compounds of formula I may be evaluated in vitro for their efficacy and potency to specifically inhibit HSL, and such evaluation may be performed as described below.

Assays

Hormone-sensitive lipase (HSL) [classified as a triacylglycerol lipase, EC 3.1.1.3, in accordance with the Recommendations of the Nomenclature Committee of the International Union of Biochemistry and Molecular Biology (available on the World Wide Web at the following address: http://www.chem.qmw.ac.uk/iubmb/enzyme/)]

Materials. The Hormone-sensitive lipase employed was provided by Dr. Cecilia Holm of Lund University, Sweden, or produced and purified by Novo Nordisk (NN) using the reagents and protocols employed by Dr. Holm. The substrates used are: ³H-labeled triolein (TO) from Amersham, Buckinghamshire, U.K. cat No. TRA191; 5-20 Ci/mmol dissolved in toluene, triolein (Sigma, Cat. No. T-1740) and mono-oleoyl-2-O-mono-oleylglycerol (MOME) synthesized at (NN) using the protocols provided by Dr. Cecilia Holm, Lund University, Sweden. Phosphatidyl choline (PC) and phosphatidyl inositol (PI) are from Sigma (St Luis Mo., cat. Nos. P-3556 and P-5954, respectively). All other reagents are of commercial grade and obtained from various commercial sources.

Methods. The preparation of the substrates is as follows:

TO: 30 μl PC:PI (20 mg/ml solution of PC:PI 3:1 prepared in chloroform)+128 μl cold TO+15 μl ³H-TO

MOME: 30 μl PC:PI+100 μl cold MOME+200 μl ³H-MOME

They are then evaporated under a gentle stream of N₂ followed by 20-30 minutes in a Speedvac™ apparatus to ensure the absence of residual solvent.

The samples are prepared as follows:

Blank: 100 μl PED-BSA

Samples/controls: 78 μl PED-BSA+20 μl inhibitor (various concentrations)+2 μl HSL (undiluted 0.12 mg/ml). If no inhibitors are being tested, the reaction contains 98 μl PED-BSA+2 μl HSL.

The samples are put on ice and then sonicated as follows:

With TO as substrate, add 2 ml potassium buffer (0.1M potassium phosphate, pH 7) and then sonicate twice for 1 minute, with a 1-minute interval between cycles. Add an additional 1 ml potassium buffer and sonicate 4 times for 30 seconds, with a 30 second interval in between cycles. To finish, add 1 ml BSA 20%-in potassium buffer, mix by inversion and store on ice until use.

With MOME as substrate: add 2 ml potassium buffer and then sonicate twice for 1 minute, with a 1 minute interval between cycles. Add 1.6 ml potassium buffer and sonicate 4 times for 30 seconds, with a 30-second interval between cycles. To finish, add 0.4 ml BSA 20%-buffer, mix by inversion and store on ice until use.

The enzyme reaction is run at 37° C. The substrate mix is added to the various reactions at 10 second intervals, incubated for 30 minutes in a water bath and then stopped with 3.25 ml of a solution containing CH₃OH:CHCl₃:heptane (10:9:7). After the addition of 1.05 ml potassium buffer pH10.5, the sample is vortexed thoroughly and centrifuged for 20 min at 2000 r.p.m. One ml of the upper phase is added to 10 ml scintillation fluid, and mixed thoroughly by shaking.

The samples are then counted, 5 min per vial, in a scintillation counter.

Lipoprotein Lipase (LPL) [Classified as EC 3.1.1.34 in accordance with the Recommendations of the Nomenclature Committee of the International Union of Biochemistry and Molecular Biology (available on the World Wide Web at the following address: http://www.chem.qmw.ac.uk/iubmb/enzyme/)]

Materials. The lipoprotein lipase employed was obtained from Dr. Gunilla Olivecrona of Ume\dot{a} University, Sweden. ³H-triolein (TO) from Amersham, Buckinghamshire, U.K., cat No. TRA191; 5-20 Ci/mmol dissolved in toluene. Intralipid is from Kabi-Fresenius). Apo CII was obtained from PMSF-treated plasma from overnight fasted rats. All other reagents are of commercial grade and obtained from various commercial sources.

Methods. The preparation of the substrate is as follows:

25 μl of ³H-Triolein evaporated under a gentle stream of N₂ in a 27° C. room in 4 ml chromacol glass followed by the addition of 1 ml 10% intralipid. This is mixed and sonicated on ice for 5 minutes (50% pulse mode). This can be kept for 1 week at 4° C.

1. The reaction is:

100 μl assay medium (0.3M Tris-Cl, 0.2M NaCl, 0.2 mg heparin/ml and 120 mg BSA/ml (fraction V, Sigma no. 3401, pH 8.5))

4 μl labeled intralipid

5 μl APO CII

86 μl MilliQ water

All mixed in a 13×100 mm glass tube and incubated for at least 5 minutes.

2. Add 5 μl LPL (of a solution containing 500 mU/mg)

Mix and incubate for 30 minutes at 25° C.

3. The reaction is stopped with 3.25 ml of a solution containing Methanol:chloroform:heptan 10:9:7. Add 1.05 ml buffer (100 mM Boric acid, 100 mM potassium carbonate pH 10.5) and mix 10 seconds per vial.

4. Centrifuge 20 min 12000 rpm

5. Take 1 ml of the upper phase+10 ml scintillation fluid and mix

6. Scintillation count for 5 minutes per vial.

Hepatic Lipase (HL) [classified as a triacylglycerol lipase, EC 3.1.1.3, in accordance with the Recommendations of the Nomenclature Committee of the International Union of Biochemistry and Molecular Biology (available on the World Wide Web at the following address: http://www.chem.qmw.ac.uk/iubmb/enzyme/)]

Materials. The hepatic lipase employed was obtained from Dr. Howard Wong of the University of California at Los Angeles (UCLA), USA, or produced and purified using the methods and a cell line provided by Dr. Wong's laboratory. ³H-triolein (TO) from Amersham, Buckinghamshire, U.K., cat No. TRA191; 5-20 Ci/mmol dissolved in toluene and triolein (Sigma, Cat. No. T-1740). All other reagents are of commercial grade and obtained from various commercial sources.

Assay mix

25 mg olive oil

50 μl TO in a 20 ml scintillation vial and evaporate with N₂ for 5 minutes.

Add 1 ml 10% gum arabic and 1.25 ml Tris-Cl pH 8.5 and 2 ml MilliQ water.

Sonicate, on ice, 16 times for 30 seconds with a 30-second interval between cycles.

Add 2.5 ml of 5 M NaCl+2.5 ml BSA (10% in Milli Q, pH 8)+3.25 ml Milli Q

Total volume: 12.5 ml (for 80 reactions )

If an inhibitor is going to be used, it should be incubated with the 50 μl HL (of a 125 mU/ml solution) for 70 minutes at 25° C. A 5 μl aliquot from the incubated 50 μl is added to 150 μl assay mix+45 μl Milli Q™ water and mixed.

Incubate for exactly 30 minutes at 25° C.

Stop the reaction with Methanol:chloroform:heptane 10:9:7: 3.25 ml/vial, mix; then add 1.05 ml buffer (100 mM boric acid, 100 mM potassium carbonate pH 10.5), mix for 10 seconds per vial.

Centrifuge for 20 min/2000 rpm

Take 1 ml of the upper phase+10 ml scintillation fluid and mix by shaking by hand.

Scintillation count for 5 minutes per vial.

Results

The results of the inhibition assays for the specific compounds of the invention described in the working examples (vide infra) are summarized in Table 1, below.

TABLE 1

| | HSL inhibition | LPL inhibition | HL inhibition |
|---|---|---|---|
| Example No. | (IC₅₀, μM) | (% inh. at 50 μM) | (% inh. at 50 μM) |
| 1 | 0.2 | | 5 |
| 2 | 6.4 | 2 | |
| 3 | 0.36 | 0 | |
| 4 | 9.9 | 20 | |
| 5 | 0.039 | 7 | |
| 6 | 0.019 | 0 | 6 |
| 7 | 0.46 | 0 | |
| 8 | 0.03 | 1 | |
| 9 | 1.6 | 7 | |
| 10 | 0.67 | 5 | |
| 11 | >10 | 7 | |
| 12 | — | | |
| 13 | 0.045 | 3 | |
| 14 | 0.8 | 7 | |
| 15 | 1.5 | 0 | |
| 16 | 1.4 | 0 | |
| 17 | 0.003 | 0 | 3 |
| 18 | 7.7 | 0 | |
| 19 | — | | |
| 20 | 0.24 | 4 | |
| 21 | >10 | 5 | |
| 22 | 0.03 | 10 | |
| 23 | 0.04 | 4 | 4 |
| 24 | >10 | | |
| 25 | >10 | | |
| 26 | >10 | | |
| 27 | 0.09 | 5 | |
| 28 | 0.07 | 3 | |
| 29 | 2.6 | 0 | |
| 30 | 3.8 | 6 | |
| 31 | >10 | 1 | |
| 32 | >10 | 0 | |
| 33 | 0.17 | 2 | |
| 34 | >10 | | |
| 35 | >10 | 0 | |
| 36 | >10 | | |
| 37 | 3.9 | 0 | |
| 38 | 0.33 | 0 | |
| 39 | 2.5 | 2 | |
| 40 | 3.4 | 1 | |
| 41 | >10 | | |
| 42 | 0.04 | 6 | |
| 43 | >10 | 6 | |
| 44 | >10 | | |
| 45 | >10 | | |
| 46 | — | | |
| 47 | >10 | | |
| 48 | 0.05 | | |
| 49 | 0.10 | | |
| 50 | >10 | | |

General Methods

The compounds of the invention may be prepared by means of the carbazate chemistry which is well known in the art, for example in M. Salman, S. Ray Indian J. Chem. Sect. B 1981, 20, 477-479; M. Marastoni, M. Bergonzoni, F. Bortolotti, R. Tomatis Arzneim. Forsch. 1997, 47, 889-894; R. R. L. Hamer, R. C. Effland, J. T. Klein J. Heterocyclic Chem. 1988, 25, 517; M. R. Mish, F. M. Guerra, E. M. Carreira J. Am. Chem. Soc. 1997, 119, 8379-8380; W. P. Brian J. Med. Chem. 1971, 14, 1133-1134, as summarized in Schemes 1 to 3 wherein A, R¹, X¹, X² and B are as previously defined.

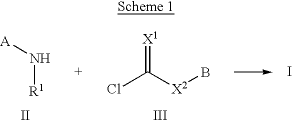

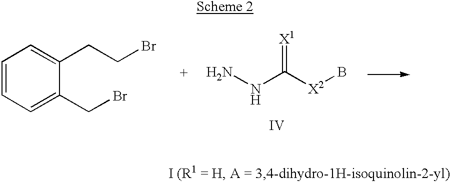

wherein X¹=O, X²=O

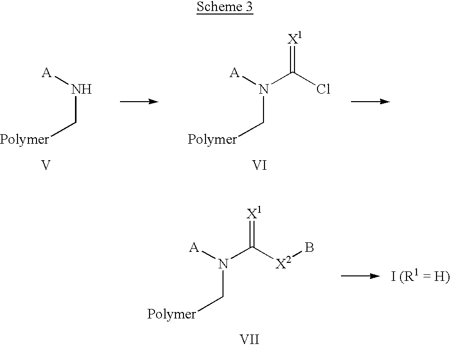

The hydrazines II may be readily obtained by published methodology, for example as described by: J. H. Biel, A. E. Drukker, T. F. Mitchell, E. P. Sprengeler, P. A. Nuhfer, A. C. Conway, A. Horita, J. Am. Chem. Soc. 1959, 81, 2805; K. Sakane, K.-i. Terayama, E. Haruki, Y. Otsuj, E. Imoto, Bull. Chem. Soc. Jpn. 1974, 47, 1297-1298; L. Landriani, D. Barlocco, G. A. Pinna, M. P. Demontis, M. M. P. Enrico, V. Anania, II Farmaco 1989, 44, 1059-1068; E. Höft, A. Rieke, Angew. Chem. 1961, 73, 1297-1298.

The chloroformates and chlorodithioformates III may be readily obtained either from commercial sources or by published methodology, for example as described by: L. N. Owen, R. Sridhar, J. Chem. Soc (C) 1970, 472475; T. Konakahara, T. Ozaki, K. Sato, B. Gold, Synthesis 1993, 103-106; A.Vigroux, M. Bergon, C. Zedde, J. Med. Chem. 1995, 38, 3983-3994.

Other compounds used as starting materials are either known compounds or compounds that can readily be prepared by methods known per se.

Preparation of the compounds of the present invention is further illustrated in the following examples, which are, however, not to be construed as limiting the invention in any way.

The structures and purities of the compounds were determined by High Performance Liquid Chromatography (HPLC), Nuclear Magnetic Resonance (NMR, Bruker 200 and 300 MHz) or Liquid Chromatography-Mass Spectrometry (LC-MS). NMR chemical shifts (8) are given in parts per million (ppm), and only selected peaks are given. Column chromatography was carried out using the technique described by W. C. Still et al., J. Org. Chem. 1978, 43, 2923-2925, on Macherey-Nagel silica gel 60 (230-400 mesh).

HPLC-Analysis:

The RP-analysis was performed using UV detection at 210 and 254 nm on a YMC 120 Å 5 μl HPLC column (50×4.0 mm i.d., C-18 silica), which was eluted at 2.8 mL/min. The column was equilibrated with a solution of trifluoroacetic acid (TFA) (0.01%) in water. After injection the sample was eluted with a linear gradient of 0% to 90% acetonitrile containing 0.01% TFA in the same aqueous buffer over a period of 10 min.

### EXAMPLES

### Example 1

### (3,4-Dihydro-1H-isoquinolin-2-yl)-carbamic Acid Phenyl Ester

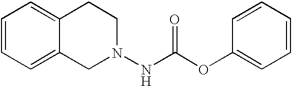

N,N-Diisopropylethylamine (2.41 mL) was added to a stirred solution of 2-(2-bromomethylphenyl)ethylbromide (1.00 g) and hydrazinecarboxylic acid phenyl ester (0.94 g) in N,N-dimethylformamide (20 mL). The reaction mixture was heated at 60° C. overnight, cooled down to room temperature and poured into water. The precipitate was isolated by suction and recrystallised from ethyl acetate/heptane yielding the title compound as a white solid.

¹H-NMR (300 MHz, CDCl₃); δ=3.06 (t, 2H), 3.30 (t, 2H), 4.16 (s, 2H), 7.03 (m, 1H), 7.17 (m, 6H), 7.36 (m, 2H).

### Example 2

### (3,4-Dihydro-1H-isoquinolin-2-yl)-carbamic acid 2-methoxy-phenyl Ester

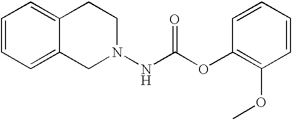

N,N-Diisopropylethylamine (2.41 mL) was added to a stirred solution of 2-(2-bromomethylphenyl)ethylbromide (1.00 g) and hydrazinecarboxylic acid 2-methoxyphenyl ester (1.13 g) in N,N-dimethylformamide (20 mL). The reaction mixture was heated at 60° C. for 3 h, cooled down to room temperature and poured into water. The precipitate was isolated by suction, stripped with acetonitrile and dried at 40° C. in vacuo overnight yielding the title compound as a white solid.

¹H-NMR (300 MHz, CDCl₃); δ=3.05 (t, 2H), 3.32 (t, 2H), 3.87 (s, 3H), 4.17 (s, 2H), 6.39 (br.s, 1H), 6.94 (m, 2H), 7.03 (m, 1H), 7.14 (m, 5H).

### Example 3

### (3,4-Dihydro-1H-isoquinolin-2-yl)-carbamic acid 4-methoxy-phenyl Ester

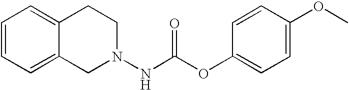

At 0° C., solution of 3,4dihydro-1H-isoquinolin-2-ylamine (0.50 g) and N,N-diisopropylethylamine (0.58 mL) in dichloromethane (10 mL) was added dropwise to a stirred solution of 4-methoxyphenyl chloroformate (0.57 mL) in dichloromethane (10 mL). After stirring overnight at room temperature the solution was extracted with water, dried over Na₂SO₄, filtered and evaporated in vacuo. The residue was recrystallised from ethyl acetate/heptane yielding the title compound as a white solid.

¹H-NMR (300 MHz, CDCl₃); δ=3.04 (t, 2H), 3.28 (t, 2H), 3.78 (s, 3H), 4.14 (s, 2H), 6.32 (br.s, 1H), 6.86 (AB-system, 2H), 7.02 (m, 1H), 7.07 (AB-system, 2H), 7.13 (m, 3H).

### Example 4

### 4-(3,4-Dihydro-1H-isoquinolin-2-ylcarbamoyloxy)-benzoic Acid Methyl Ester

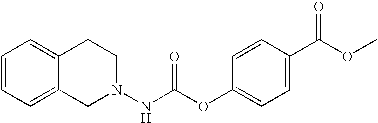

At 0° C., solution of 3,4-dihydro-1H-isoquinolin-2-ylamine (0.50 g) and N,N-diisopropylethylamine (0.58 mL) in dichloromethane (10 mL) was added dropwise to a stirred solution of 4-methoxycarbonylphenyl chloroformate (0.73 g) in dichloromethane (10 mL). After stirring overnight at room temperature the solution was extracted with water, dried over Na₂SO₄, filtered and evaporated in vacuo. The off-white residue was purified by flash column chromatography (SiO₂, ethyl acetate/heptane 30/70 followed by ethyl acetate/heptane 20/80). Recrystallisation from ethyl acetate yielded the title compound as a white solid.

¹H-NMR (300 MHz, CDCl₃); δ=3.05 (t, 2H), 3.32 (t, 2H), 3.92 (s, 3H), 4.17 (s, 2H), 6.39 (br.s, 1H), 7.04 (m, 1H), 7.16 (m, 3H), 7.25 (d, 2H), 8.06 (d, 2H).

### Example 5

### (3,4-Dihydro-1H-isoquinolin-2-yl)-carbamic acid 4-bromo-phenyl Ester

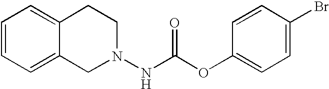

The title compound was prepared from 3,4-dihydro-1H-isoquinolin-2-ylamine hydrochloride (1.50 g) and 4-bromophenyl chloroformate (1.79 g) using the procedure as described in example 4. Purification by flash column chromatography (SiO₂, ethyl acetate/heptane 30/70), followed by recrystallisation from ethyl acetate yielded the title compound as a slightly yellow solid.

¹H-NMR (300 MHz, CDCl₃); δ=3.04 (t, 2H), 3.28 (t, 2H), 4.16 (s, 2H), 6.33 (br.s, 1H), 7.05 (m, 1H+d, 2H), 7.17 (m, 3H), 7.48 (d, 2H).

### Example 6

### (3,4-Dihydro-1H-isoquinolin-2-yl)-carbamic acid 4-chloro-phenyl Ester

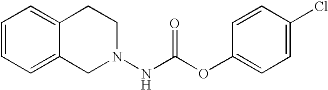

The title compound was prepared from 3,4-dihydro-1H-isoquinolin-2-ylamine hydrochloride (1.50 g) and 4-chlorophenyl chloroformate (1.43 g) using the procedure as described in example 4. Purification by flash column chromatography (SiO₂, ethyl acetate/heptane 30/70), followed by recrystallisation from ethyl acetate yielded the title compound as a slightly yellow solid.

¹H-NMR (300 MHz, CDCl₃); δ=3.04 (t, 2H), 3.29 (t, 2H), 4.15 (s, 2H), 6.35 (br.s, 1H), 7.03 (m, 1H), 7.11 (AB-system, 2H), 7.17 (m, 3H), 7.33 (AB-system, 2H).

### Example 7

### 3,4-Dihydro-1H-isoquinolin-2-yl)-carbamic Acid 4-fluoro-phenyl Ester

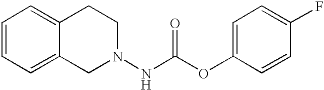

The title compound was prepared from 3,4-dihydro-1H-isoquinolin-2-ylamine (0.50 g) and 4-fluorophenyl chloroformate (0.59 g) using the procedure as described in example 4. Purification by flash column chromatography (SiO₂, ethyl acetate/heptane 30/70), followed by recrystallisation from ethyl acetate yielded the title compound as a yellow solid.

¹H-NMR (300 MHz, CDCl₃); δ=3.04 (t, 2H), 3.29 (t, 2H), 3.92 (s, 3H), 4.16 (s, 2H), 6.33 (br.s, 1 H), 6.99-7.21 (m, 8H).

### Example 8

### (3,4-Dihydro-1H-isoquinolin-2-yl)-carbamic Acid p-tolyl Ester

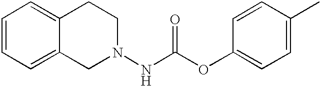

A solution of 3,4-dihydro-1H-isoquinolin-2-ylamine (0.50 g) in a few mL's of dry ether was added slowly to a stirred solution of p-tolyl chloroformate (0.57 g) in dry ether (15 mL), followed by the addition of pyridine (0.27 g) dissolved in a few mL's of dry ether. After stirring for 5 min. at room temperature, dichloromethane was added and the solution was extracted with water. The organic layer was dried over Na₂SO₄, filtered and evaporated in vacuo. Recrystallisation of the residue from ethyl acetate yielded the title compound as a white solid.

¹H-NMR (300 MHz, CDCl₃); δ=2.33 (s, 3H), 3.03 (t, 2H), 3.27 (t, 2H), 4.14 (s, 2H), 6.36 (br.s, 1H), 7.02 (m, 3H), 7.15 (m, 5H).

### Example 9

### (3,4-Dihydro-1H-isoquinolin-2-yl)-carbamic Acid o-tolyl Ester

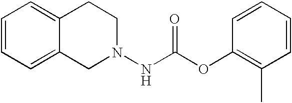

o-Tolyl chloroformate (0.43 g) was added to a stirred solution of 3,4-dihydro-1H-isoquinolin-2-ylamine hydrochloride (0.50 g) and N,N-diisopropylethylamine (0.44 mL) in dichloromethane (10 mL). The reaction mixture was stirred for 1 h at 0° C. followed by stirring overnight at room temperature. Extra dichloromethane was added and the solution was extracted with water. The organic layer was dried over Na₂SO₄, filtered and evaporated in vacuo. Flash column chromatography (SiO₂, ethyl acetate/heptane 30/70), followed by recrystallisation from ethyl acetate yielded the title compound as a white solid.

¹H-NMR (300 MHz, CDCl₃); δ=2.25 (s, 3H), 3.05 (t, 2H), 3.31 (t, 2H), 4.18 (s, 2H), 6.37 (br.s, 1H), 7.02-7.22 (m, 8H).

### Example 10

### (3,4-Dihydro-1H-isoquinolin-2-yl)-carbamic Acid m-tolyl Ester

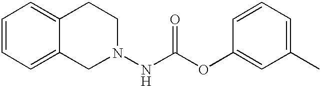

The title compound was prepared from 3,4-dihydro-1H-isoquinolin-2-ylamine hydrochloride (0.50 g) and m-tolyl chloroformate (0.43 g) using the procedure as described in example 9.

¹H-NMR (300 MHz, CDCl₃); δ=2.36 (s, 3H), 3.05 (t, 2H), 3.31 (t, 2H), 4.17 (s, 2H), 6.33 (br.s, 1H), 6.93-7.25 (m, 8H).

### Example 11

### (3,4-Dihydro-1H-isoquinolin-2-yl)-carbamic Acid 2.6-dichloro-phenyl Ester

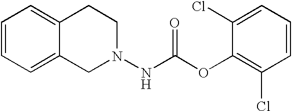

2,6-Dichlorophenyl chloroformate (0.57 g) was added to a stirred solution of 3,4-dihydro-1H-isoquinolin-2-ylamine hydrochloride (0.50 g) and N,N-diisopropylethylamine (0.44 mL) in dichloromethane (10 mL). The reaction mixture was stirred for 1 h at 0° C. followed by stirring for 3 d at room temperature. Extra dichloromethane was added and the solution was extracted with water. The organic layer was dried over Na₂SO₄, filtered and evaporated in vacuo. Flash column chromatography (SiO₂, ethyl acetate/heptane 30/70), followed by recrystallisation from ethyl acetate yielded the title compound as a white solid.

¹H-NMR (200 MHz, CDCl₃); δ=3.04 (t, 2H), 3.33 (t, 2H), 4.20 (s, 2H), 6.53 (br.s, 1H), 7.02-7.42 (m, 7H).

### Example 12

### (3,4-Dihydro-1H-isoquinolin-2-yl)-carbamic Acid 2,4-dichloro-phenyl Ester

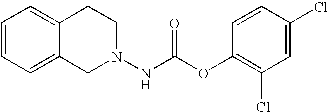

2,4-Dichlorophenyl chloroformate (0.90 g) and triethylamine (0.81 g) were added respectively to a stirred suspension of 3,4-dihydro-1H-isoquinolin-2-yiamine hydrochloride (0.79 g) in dichloromethane (25 mL). The reaction mixture was stirred for 5 min at 0° C. followed by stirring at room temperature. After 2.5 h an extra equivalent of 2,4-dichlorophenyl chloroformate was added, followed by a third equivalent after another 2 h. After stirring overnight the solution was extracted with water. The organic layer was dried over Na₂SO₄, filtered and evaporated in vacuo. Flash column chromatography (SiO₂, ethyl acetate/heptane 30/70), followed by recrystallisation from ethyl acetate yielded the title compound as a white solid.

¹H-NMR (300 MHz, CDCl₃); δ=3.06 (t, 2H), 3.31 (t, 2H), 4.17 (s, 2H), 6.47 (br.s, 1H), 7.03 (m, 1H), 7.10-7.28 (m, 5H), 7.43 (d, 1H).

### Example 13

### (3,4-Dihydro-1H-isoquinolin-2-yl)-carbamic Acid 4-trifluoromethyl-phenyl Ester

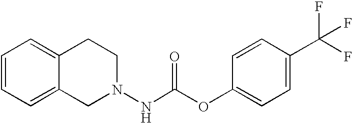

The title compound was prepared from 3,4-dihydro-1H-isoquinolin-2-ylamine (0.50 g) and 4-trifluoromethylphenyl chloroformate (0.76 g) using the procedure as described in example 8.

¹H-NMR (300 MHz, CDCl₃); δ=3.04 (t, 2H), 3.30 (t, 2H), 4.15 (s, 2H), 7.03 (m, 1H), 7.15 (m, 3H), 7.39 (d, 2H), 7.63 (d, 2H).

### Example 14

### (3,4-Dihydro-1H-isoquinolin-2-yl)-carbamic Acid 2-bromo-phenyl Ester

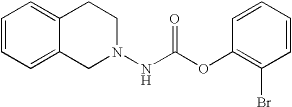

3,4-Dihydro-1H-isoquinolin-2-ylamine hydrochloride (0.79 g) and triethylamine (0.81 g) were added respectively to a stirred solution of 2-bromophenyl chloroformate (0.94 g) in dichloromethane (25 mL). After stirring for 0.5 h water was added and both layers were separated. The organic layer was dried over Na₂SO₄, filtered and evaporated in vacuo yielding a white solid. Flash column chromatography (SiO₂, ethyl acetate), followed by recrystallisation from ethyl acetate and chloroform yielded the title compound as a white solid.

¹H-NMR (300 MHz, CDCl₃); δ=3.06 (t, 2H), 3.32 (t, 2H), 4.18 (s, 2H), 6.50 (br.s, 1H), 7.00-7.35 (m, 7H), 7.58 (dd, 1H).

### Example 15

### (3,4-Dihydro-1H-isoquinolin-2-yl)-carbamic Acid 2-fluoro-phenyl Ester

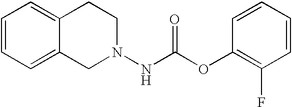

The title compound was prepared from 3,4-dihydro-1H-isoquinolin-2-ylamine hydrochloride (0.79 g) and 2-fluorophenyl chloroformate (0.70 g) using the procedure as described in example 14.

¹H-NMR (300 MHz, CDCl₃); δ=3.03 (t, 2H), 3.29 (t, 2H), 4.17 (s, 2H), 6.48 (br.s, 1H), 7.03 (m, 1H), 7.08-7.27 (m, 7H).

### Example 16

### (6,7-Dimethoxy-3.4-dihydro-1H-isoquinolin-2-yl)-carbamic Acid 4-chloro-phenyl Ester

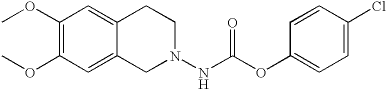

Triethylamine (0.17 mL) was added to a stirred suspension of 6,7-dimethoxy-3,4-dihydro-1H-isoquinolin-2-ylamine hydrochloride (0.30 g) and 4-chlorophenyl chloroformate (0.23 g) in dichloromethane (10 mL). After stirring for 3 d at room temperature additional dichloromethane was added and the solution was extracted with water. The water layer was extracted with dichloromethane and the combined organic layers were dried over Na₂SO₄, filtered and evaporated in vacuo. Recrystallisation from ethyl acetate yielded the title compound as a white solid.

¹H-NMR (300 MHz, CDCl₃); δ=2.96 (t, 2H), 3.27 (t, 2H), 3.85 (s, 3H), 3.86 (s, 3H), 4.07 (s, 2H), 6.38 (br.s, 1H), 6.51 (s, 1H), 6.62 (s, 1H), 7.12 (AB-system, 2H), 7.33 (AB-system, 2H).

### Example 17

### (3,4-Dihydro-1H-isoquinolin-2-yl)-carbamic Acid 4-tert-butoxycarbonylamino-phenyl Ester

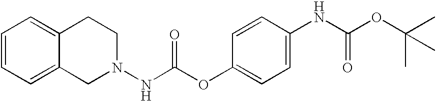

(4-Hydroxyphenyl)-carbamic acid tert-butyl ester (355 mg) and pyridine (284 mg) were dissolved in dichloromethane (20 mL). A solution of triphosgene (168 mg) in dichloromethane (10 mL) was added. After stirring for 1 h at 0° C., a solution of 3,4-dihydro-1H-isoquinolin-2-ylamine hydrochloride (315 mg) and triethylamine (172 mg) in dichloromethane (10 mL) was added. The cooling bath was removed and the solution was stirred overnight at room temperature. A small amount of triethylamine was added and stirring was continued for 1 h. The solution was extracted with water (2×), dried over Na₂SO₄, filtered and evaporated in vacuo. Purification by flash column chromatography (SiO₂, ethyl acetate/heptane 30/70) followed by recrystallisation from ethyl acetate/heptane yielded the title compound as a white solid.

¹H-NMR (300 MHz, CDCl₃); δ=1.52 (s, 9H), 3.04 (t, 2H), 3.28 (t, 2H), 4.14 (s, 2H), 6.32 (br.s, 1H), 6.47 (br.s, 1H), 7.00-7.18 (m, 6H), 7.33 (d, 2H).

LC-MS (electrospray) m/z: 384 (M+H)⁺, 767 (2M+H)⁺.

### Example 18

### (3,4-Dihydro-1H-isoquinolin-2-yl)-carbamic Acid 4-amino-phenyl Ester Hydrochloride

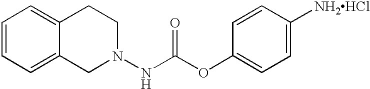

A solution of (3,4-dihydro-1H-isoquinolin-2-yl)-carbamic acid 4-tert-butoxycarbonylamino-phenyl ester (32 mg) in a mixture of tetrahydrofuran/formic acid/1N hydrochloric acid (5 mL) was stirred overnight at room temperature. The solvent was evaporated under reduced pressure and the residue was recrystallised from ethanol yielding the title compound as a white solid.

¹H-NMR (300 MHz, MeOH-d₄); δ=3.05 (t, 2H), 3.19 (t, 2H), 4.07 (s, 2H), 7.06 (m, 1H), 7.12 (m, 3H), 7.33 (AB-system, 2H), 7.42 (AB-system, 2H).

LC-MS (electrospray) m/z: 284.0 (M+H)⁺, 589.3 (2M+Na)⁺.

### Example 19

### 4-(3,4-Dihydro-1H-isoquinolin-2-ylcarbamoyloxy)-benzoic Acid Benzyl Ester

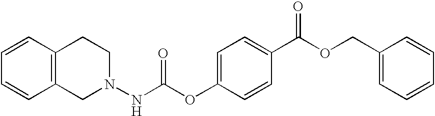

Pyridine (237 mg) was added carefully to a stirred solution of 4-chlorocarbonyloxy-benzoic acid benzyl ester (1.31 g) and 3,4-dihydro-1H-isoquinolin-2-ylamine hydrochloride (554 mg) in dichloromethane (10 mL). After stirring for 24 h at room temperature extra dichloromethane was added and the solution was extracted with water. The organic layer was dried over Na₂SO₄, filtered and evaporated in vacuo. The residue was purified by flash column chromatography (SiO₂, ethyl acetate/heptane 20/80) yielding the title compound as a white solid.

¹H-NMR (300 MHz, CDCl₃); δ=3.06 (t, 2H), 3.29 (t, 2H), 4.16 (s, 2H), 5.35 (s, 2H), 6.45 (br.s, 1H), 7.03 (m, 1H), 7.16 (m, 3H), 7.23 (d, 2H), 7.33-7.47 (m, 5H), 8.08 (d, 2H),

LC-MS (electrospray) m/z: 403 (M+H)⁺, 827 (2M+Na)⁺.

### Example 20

### (3,4-Dihydro-1H-isoquinolin-2-yl)-carbamic Acid 4-propyl-phenyl Ester

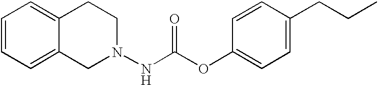

At 0° C., 4-n-propylphenyl chloroformate (0.79 g) and triethylamine (0.81 g) were added respectively to a stirred suspension of 3,4-dihydro-1H-isoquinolin-2-ylamine hydrochloride (0.79 g) in dichloromethane (25 mL). The cooling bath was removed and the reaction mixture was stirred overnight at room temperature. Another equivalent of 4-n-propylphenyl chloroformate was added and stirring was continued for another 2.5 h. The solution was extracted with water, dried over Na₂SO₄, filtered and evaporated in vacuo, yielding a slightly yellow powder. The residue was purified by flash column chromatography (SiO₂, ethyl acetate/heptane 30/70). Recrystallisation from ethyl acetate yielded the title compound as a white solid.

¹H-NMR (300 MHz, CDCl₃); δ=0.93 (t, 3H), 1.63 (m, 2H), 2.56 (t, 2H), 3.03 (t, 2H), 3.28 (t, 2H), 4.16 (s, 2H), 6.34 (br.s, 1H), 7.02 (m, 3H), 7.15 (m, 5H).

### Example 21

### (3,4-Dihydro-1H-isoquinolin-2-yl)-methyl-carbamic Acid 4-chloro-phenyl Ester

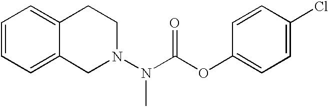

Triethylamine (0.42 mL) was added to a stirred suspension of (3,4-dihydro-1H-isoquinolin-2-yl)-methylamine hydrochloride (200 mg) in dichloromethane (10 mL), followed by a dropwise addition of 4-chlorophenyl chloroformate (211 mg). After stirring overnight at room temperature, extra dichloromethane was added and the solution was extracted with water. The organic layer was dried over Na₂SO₄, filtered and evaporated in vacuo. The residue was purified by flash column chromatography (SiO₂, ethyl acetate/heptane 10/90) yielding the title compound as a white solid.

¹H-NMR (300 MHz, CDCl₃); δ=2.8-3.5 (br.m, 7H), 4.24.4 (br.d, 2H), 7.00-7.18 (m, 6H), 7.30 (d, 2H).

### Example 22

### (1-Methyl-3,4-dihydro-1H-isoquinolin-2-yl)-carbamic Acid 4chloro-phenyl Ester

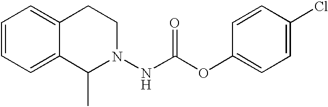

Triethylamine (0.84 mL) was added dropwise to a stirred suspension of 1-methyl-3,4-dihydro-1H-isoquinolin-2-ylamine hydrochloride (0.60 g) and 4-chlorophenyl chloroformate (0.63 g) in dichloromethane (20 mL). After stirring for 2 h at room temperature, extra dichloromethane was added and the solution was extracted twice with water. The organic layer was dried over Na₂SO₄, filtered and evaporated in vacuo. The residue was recrystallised twice from ethyl acetate yielding the title compound as a white solid.

¹H-NMR (300 MHz, CDCl₃); δ=1.55 (d, 3H), 2.83-3.49 (m, 4H), 4.15 (br.s, 1H), 6.30 (br.s 1 H), 7.07-7.22 (m, 6H), 7.32 (d, 2H).

### Example 23

### (3-Methyl-3,4-dihydro-1H-isoquinolin-2-yl)-carbamic Acid 4-chloro-phenyl Ester

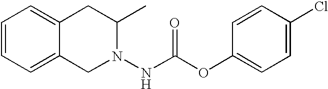

At 0° C., N,N-diisopropylethylamine (781 mg) was added to a stirred solution of 3-methyl-3,4-dihydro-1H-isoquinolin-2-ylamine hydrochloride (600 mg) and 4-chlorophenyl chloroformate (577 mg) in dichloromethane (10 mL). The cooling bath was removed and stirring was continued for 3 h at room temperature. The reaction mixture was extracted with water (2×), dried over Na₂SO₄, filtered and evaporated in vacuo. Purification of the residue by flash column chromatography yielded the title compound as a white solid.

¹H-NMR (300 MHz, CDCl₃); δ=1.31 (d, 3H), 2.80 (dd, 1H), 2.88 (dd, 1H), 3.32 (br.s, 1H), 4.17 (d, 1H), 4.31 (br.d, 1 H), 6.21 (br.s, 1H), 7.06 (m, 4H), 7.18 (m, 2H), 7.30 (d, 2H).

LC-MS (electrospray) m/z: 317 (M+H)⁺.

### Examples 24-47

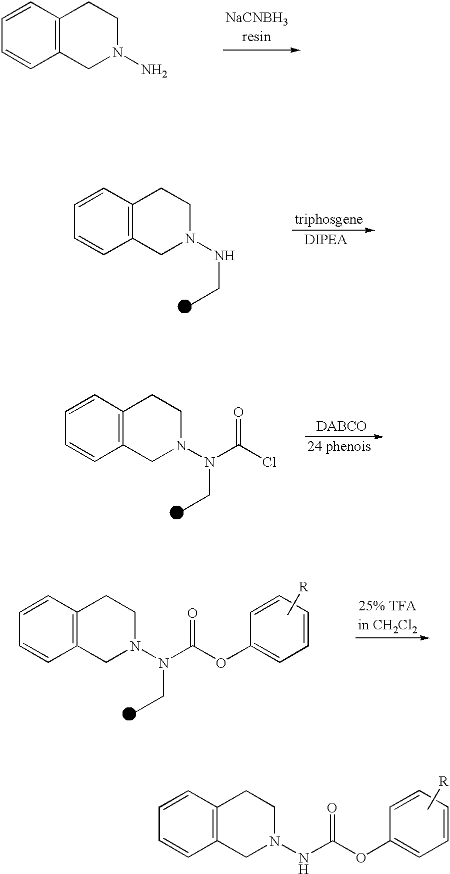

The 4-(4-formyl-3,5-dimethoxyphenoxy)butyryl aminomethyl polystyrene resin (loading 0.62 mMol/g, 3.0 g) was swollen in dichloromethane for 0.5 min. The solvent was removed and a solution of 3,4-dihydro-1H-isoquinolin-2-ylamine hydrochloride (3.43 g) in a mixture of N-methylpyrolidinone (30 mL) and water (1.5 mL) was added, followed by sodium cyanoborohydride (1.73 g) in N-methylpyrolidinone (10 mL) and acetic acid (4 mL). The reaction mixture was shaken overnight at room temperature. The solvent was removed and the resin was washed with N-methylpyrolidinone (3×30 mL), dichloromethane (3×30 mL) and methanol (3×30 mL), followed by drying overnight at 40° C. in a vacuum oven. The resin (2.63 g) was swollen in dichloromethane for 0.5 min. Triphosgene (1.45 g) dissolved in dichloromethane (30 mL), and N,N-diisopropylethylamine (2.11 g) were added respectively. The reaction mixture was shaken for 4 h at room temperature, washed with dichloromethane (3×) and diethyl ether (3×) and dried overnight at 40° C. in a vacuum oven. A suspension of the resin was made in a 1:1 mixture of 1,2-dichloropropane and N-methylpyrolidinone (50 mL), which was divided equally into 24 reaction vessels. The solvent was removed and the resin was washed with N,N-dimethylformamide. 1,4-Diazabicyclo[2.2.2]octane (10 equivalents for each reaction vessel) dissolved in N,N-dimethylformamide (25 mL) was divided equally into the 24 reaction vessels. The reaction mixtures were shaken for 0.5 h at room temperature. The 24 different phenols were each dissolved in N,N-dimethylformamide (1 mL) and added to the 24 reaction vessels. After shaking overnight the solvent was removed and the resin was washed with N,N-dimethylformamide (5×) and dichloromethane (10×) respectively. The vessels were each treated with dichloromethane (1.5 mL) and trifluoroacetic acid (0.5 mL) for 1 h. The filtrates were collected and evaporated in vacuo yielding 24 compounds as solids or as highly viscous oils. The compounds were analysed by LC-MS in which all 24 compounds showed the expected molecular ion.

| EXAMPLE | PRODUCT | Formula | MW | LC-MS |
|---|---|---|---|---|
| EXAMPLE 24 | 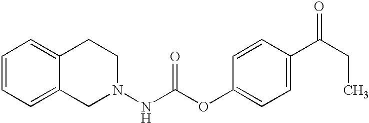 | C₁₉H₂₀N₂O₃ | 324.38 | 325 |
| EXAMPLE 25 | 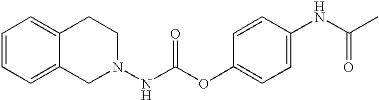 | C₁₈H₉N₃O₃ | 325.37 | 326 |
| EXAMPLE 26 | 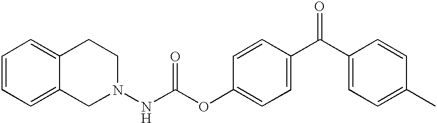 | C₂₄H₂₂N₂O₃ | 386.45 | 388 |
| EXAMPLE 27 | 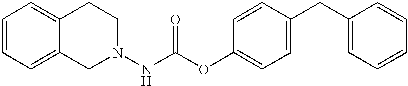 | C₂₃H₂₂N₂O₂ | 358.44 | 359 |
| EXAMPLE 28 | 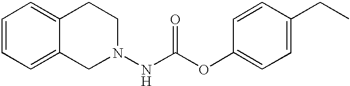 | C₁₈H₂₀N₂O₂ | 296.37 | 297 |
| EXAMPLE 29 | 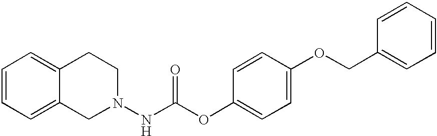 | C₂₃H₂₂N₂O₃ | 374.44 | 376 |
| EXAMPLE 30 | 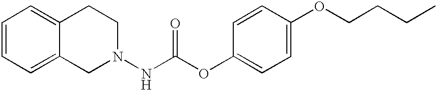 | C₂₀H₂₄N₂O₃ | 340.42 | 341 |
| EXAMPLE 31 | 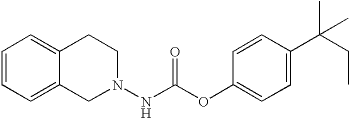 | C₂₁H₂₆N₂O₂ | 338.45 | 340 |
| EXAMPLE 32 | 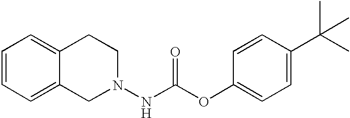 | C₂₀H₂₄N₂O₂ | 324.42 | 325 |
| EXAMPLE 33 | 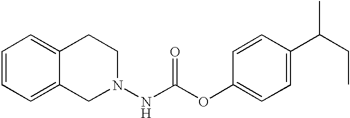 | C₂₀H₂₄N₂O₂ | 324.42 | 325 |
| EXAMPLE 34 | 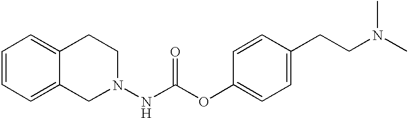 | C₂₀H₂₅N₃O₂ | 339.44 | 340 |
| EXAMPLE 35 | 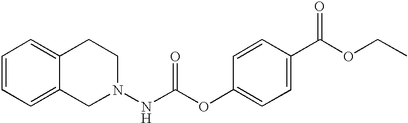 | C₁₉H₂₀N₂O₄ | 340.38 | 341 |
| EXAMPLE 36 | 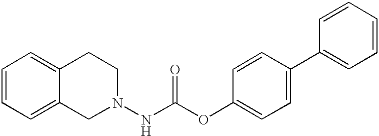 | C₂₂H₂₀N₂O₂ | 344.41 | 345 |
| EXAMPLE 37 | 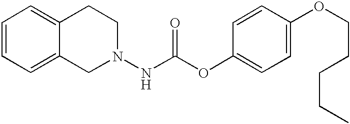 | C₂₁H₂₆N₂O₃ | 354.45 | 355 |
| EXAMPLE 38 | 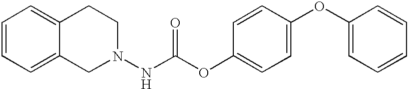 | C₂₂H₂₀N₂O₃ | 360.41 | 361 |
| EXAMPLE 39 | 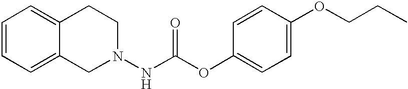 | C₁₉H₂₂N₂O₃ | 326.39 | 327 |
| EXAMPLE 40 | 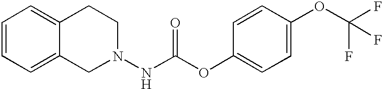 | C₁₇H₁₅F₃N₂O₃ | 352.31 | 353 |
| EXAMPLE 41 | 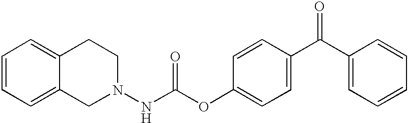 | C₂₃H₂₀N₂O₃ | 372.42 | 373 |
| EXAMPLE 42 | 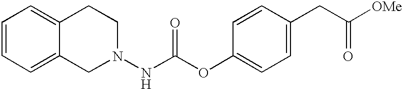 | C₁₉H₂₀N₂O₄ | 340.38 | 341 |
| EXAMPLE 43 | 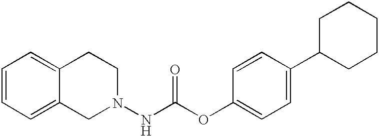 | C₁₉H₂₀N₂O₄ | 350.46 | 351 |
| EXAMPLE 44 | 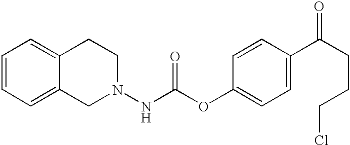 | C₂₀H₂₁ClN₂O₃ | 372.85 | 373 |
| EXAMPLE 45 | 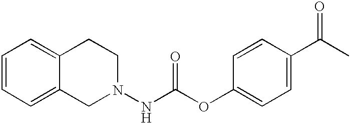 | C₁₈H₁₈N₂O₃ | 310.35 | 311 |
| EXAMPLE 46 | 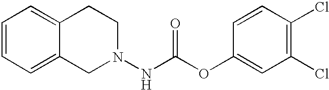 | C₁₆H₁₄Cl₂N₂O₂ | 337.20 | 337 |
| EXAMPLE 47 | 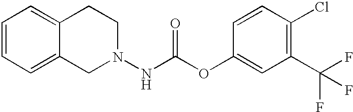 | C₁₇H₁₄ClF₃N₂O₂ | 370.76 | 371 |

### Example 48

### Piperidin-1-yl-carbamic Acid 4-chloro-phenyl Ester

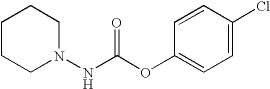

4-Chlorophenyl chloroformate (2.84 g) and triethylamine (2.09 mL) were added respectively to a stirred solution of N-aminopiperidine (1.50 g) in dichloromethane (30 mL). After stirring for 3 d at room temperature the solution was extracted with water. The organic layer was dried over Na₂SO₄, filtered and evaporated in vacuo. Purification by flash column chromatography (ethyl acetate/heptane 20/80) yielded the title compound as a white solid.

¹H-NMR (300 MHz, CDCl₃); δ=1.42 (m, 2H), 1.72 (m, 4H), 2.83 (m, 4H), 5.96 (br.s, 1H), 7.08 (d, 2H), 7.28 (d, 2H).

LC-MS (electrospray) m/z: 255 (M+H)⁺.

### Example 49

### Morpholin-4-yl-carbamic Acid 4-chloro-phenyl Ester

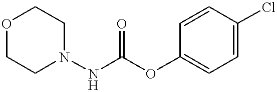

4-Chlorophenyl chloroformate (1.15 g) was added slowly to a stirred solution of N-aminomorpholine (613 mg) and pyridine (475 mg) in dichloromethane. After stirring overnight dichloromethane was added and the solution was extracted with water, dried over Na₂SO₄, filtered and evaporated in vacuo. Recrystallisation of the residue from ethyl acetate/heptane yielded the title compound as a white solid.

¹H-NMR (300 MHz, CDCl₃); δ=2.92 (m, 4H), 3.81 (m, 4H), 6.14 (br.s, 1H), 7.08 (AB-system, 2H), 7.32 (AB-system, 2H).

LC-MS (electrospray) m/z: 257 (M+H)⁺.

### Example 50

### (3,4-Dihydro-1H-isoquinolin-2-yl)-dithiocarbamic Acid Phenyl Ester

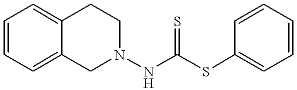

Phenyl chlorodithioformate (0.53 g) and 4-dimethylaminopyridine (0.66 g) were added to a stirred solution of 3,4-dihydro-1H-isoquinolin-2-ylamine hydrochloride (0.50 g) in N,N-dimethylformamide (10 mL). The reaction mixture was stirred for 5 d at room temperature. Flash column chromatography (SiO₂, ethyl acetate/heptane 30/70), followed by recrystallisation from ethyl acetate/heptane yielded the title compound as a white solid.

¹H-NMR (300 MHz, DMSO-d₆); δ=2.82-3.22 (m, 3H), 3.32 (br.s, 1H), 4.03 (br. AB-system, 1H), 4.19 (br. AB-system, 1H), 7.16 (m, 4H), 7.43 (s, 5H), 11.27 (s, 1H).

LC-MS (electrospray) m/z: 301 (M+H)⁺.

1. A compound of the general formula I 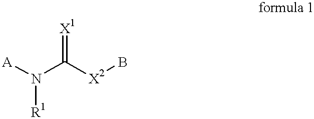
wherein
A is a nitrogen containing ring system attached through the nitrogen atom, which nitrogen containing ring system is optionally substituted with one or more substituents independently selected from halogen, hydroxy, amino, oxy, cyano, nitro, C₁₋₆-alkyl and C₁₋₆-alkoxy;
wherein each of the C₁₋₆-alkyl or C₁₋₆-alkoxy may optionally be substituted with one or more substituents independently selected from hydroxy, halogen, amino, cyano and nitro;
R¹ is hydrogen or C₁₋₆-alkyl;
X¹ is —S— or —O—;
X² is —S— or —O—;
B is aryl optionally substituted with one or more substituents independently selected from halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl and —C₁₋₆-alkyl-NR²⁰—C₁₋₆-alkyl;
wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;
R²⁰ is hydrogen or C₁₋₆-alkyl;
or B is 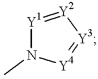
wherein
Y¹ is —C(R²¹)═ or —N═;
Y² is —C(R²²)═ or —N═;
Y³ is —C(R²³)═ or —N═;
Y⁴ is —C(R²⁴)═ or —N═;
wherein R²¹, R²², R²³ and R²⁴ independently are hydrogen, halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁵—C(═O)—C₁₋₆-alkyl, —NR²⁵—C(═O)—C₁₋₆-alkoxy, —NR²⁵—C₁₋₆-alkyl or —C₁₋₆-alkyl-NR²⁵—C₁₋₆-alkyl;
wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;
R²⁵ is hydrogen or C₁₋₆-alkyl;
or a pharmaceutically acceptable salt thereof.

2. The compound according to claim 1, wherein A is 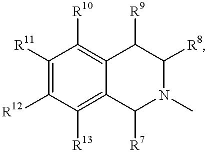
wherein R⁷, R⁸ and R⁹ independently are hydrogen or C₁₋₆-alkyl;
R¹⁰, R¹¹, R¹² and R¹³ independently are hydrogen, halogen, C₁₋₆-alkyl or C₁₋₆-alkoxy.

3. The compound according to claim 1, wherein A is 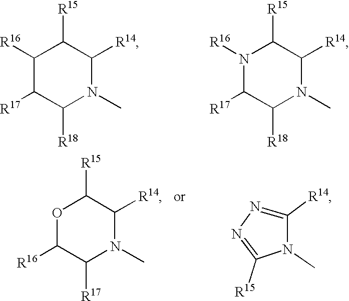
wherein R¹⁴, R¹⁵, R¹⁶, R¹⁷ and R¹⁸ independently are hydrogen, halogen, C₁₋₆-alkyl or C₁₋₆-alkoxy.

4. The compound according to any one of the preceding claims, wherein X¹ is —O—, and X² is —O—.

5. The compound according to any one of the preceding claims, wherein R¹ is hydrogen or methyl.

6. The compound according to any one of the preceding claims, wherein B is 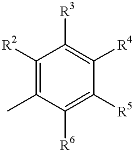
wherein
R², R³, R⁴, R⁵ and R⁶ independently of each other are hydrogen, halogen, hydroxy, amino, cyano, nitro, C₁₋₆-alkyl, C₁₋₆-alkoxy, C₁₋₆-alkylcarbonyl, C₁₋₆-alkoxycarbonyl, C₃₋₁₀-cycloalkyl, aryl, aryl-C₁₋₆-alkyl, aryl-C₁₋₆-alkoxy, aryl-C₁₋₆-alkoxycarbonyl, arylcarbonyl, —NR²⁰—C(═O)—C₁₋₆-alkyl, —NR²⁰—C(═O)—C₁₋₆-alkoxy, —NR²⁰—C₁₋₆-alkyl or —C₁₋₆-alkyl-NR²⁰—C₁₋₆-alkyl;
wherein each of the C₁₋₆-alkyl, C₁₋₆-alkoxy, C₃₋₁₀-cycloalkyl or aryl may optionally be substituted with one or more substituents independently selected from hydroxy, halogen, amino, cyano and nitro;
R²⁰ is hydrogen or C₁₋₆-alkyl.

7. The compound according to any one of the preceding claims, wherein B is

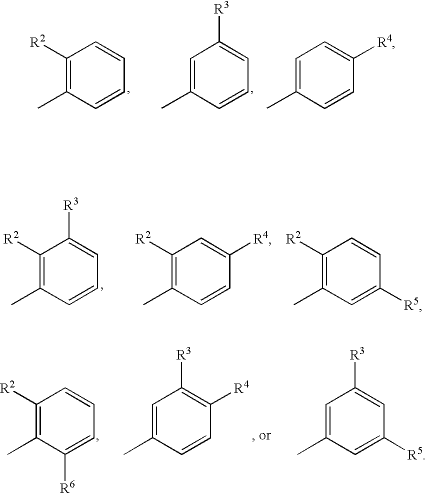

8. The compound according to claim 7, wherein B is 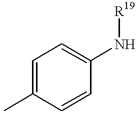
wherein R¹⁹ is hydrogen, C₁₋₆-alkyl, C₁₋₆-alkylcarbonyl, C₁₋₆-alkoxycarbonyl, aryl-C₁₋₆-alkyl, aryl-C₁₋₆-alkoxycarbonyl or arylcarbonyl.

9. The compound according to any one of the preceding claims, selected from 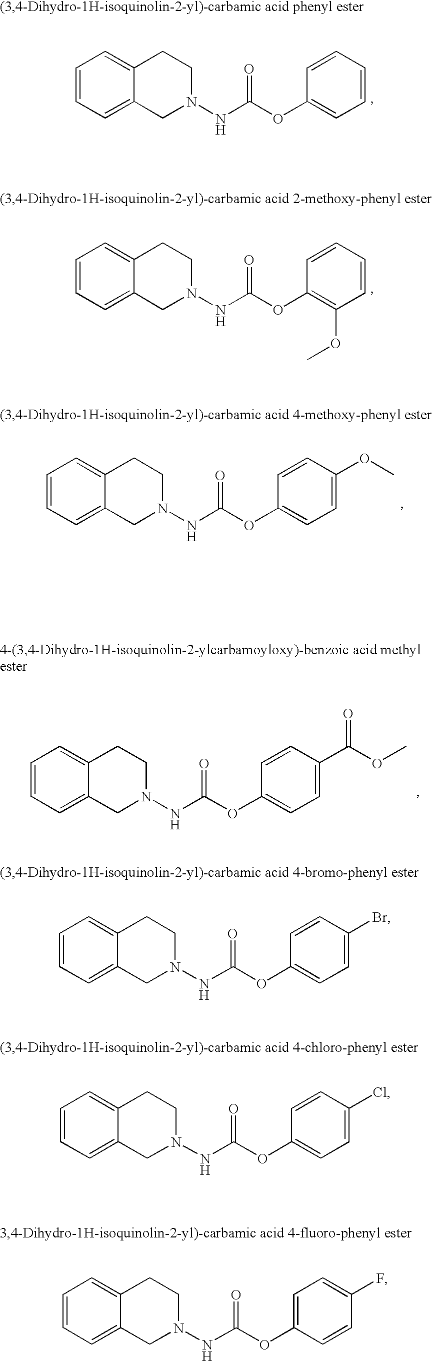
and pharmaceutically acceptable salts thereof.

10. A pharmaceutical composition comprising, as an active ingredient, a compound as defined in any one of the preceding claims, or a pharmaceutically acceptable salt thereof, together with a pharmaceutically acceptable carrier or diluent.

11. The composition according to claim 10 in unit dosage form, comprising from about 0.05 mg to about 2000 mg, preferably from about 0.1 mg to about 500 mg of the compound according to any one of claims 1-9 or pharmaceutically acceptable salt thereof.

12. A pharmaceutical composition for specifically inhibiting the lipolytic activity of HSL, said composition comprising, as an active ingredient, a compound according to any one of claims 1-9, or a pharmaceutically acceptable salt thereof, together with a pharmaceutically acceptable carrier or diluent.

13. A pharmaceutical composition according to any one of claims 10-12 for oral, nasal, transdermal, pulmonary or parenteral administration.

14. Use of a compound that specifically inhibits the lipolytic activity of HSL, or a pharmaceutically acceptable salt thereof, for the preparation of a medicament for the treatment of a disorder where a decreased level of plasma FFA is desired.

15. The use according to claim 14, wherein said disorder where a decreased level of plasma FFA is desired is diabetes type 2, insulin resistance, impaired glucose tolerance, hyperglycemia, dyslipidemia, or abnormalities of lipoprotein metabolism.

16. The use according to claim 14 or 15, wherein said compound that specifically inhibits lipolytic activity of HSL is a compound according to any one of claims 1-9 or a pharmaceutically acceptable salt thereof.

17. Use of a compound according to any one of claims 1-9, or a pharmaceutically acceptable salt thereof, for the preparation of a medicament.

18. A method of treating a disorder in a mammal where a decreased level of plasma FFA is desired, said method comprising administering to said mammal an effective amount of a compound that specifically inhibits the lipolytic activity of HSL, or a pharmaceutically acceptable salt thereof.

19. The method according to claim 18, wherein said disorder where a decreased level of plasma FFA is desired is diabetes type 2, insulin resistance, impaired glucose tolerance, hyperglycemia, dyslipidemia, or abnormalities of lipoprotein metabolism.

20. The method according to claim 18 or 19, wherein said compound that specifically inhibits lipolytic activity of HSL is a compound according to any one of claims 1-9 or a pharmaceutically acceptable salt thereof.

21. The method according to any one of claims 18-20, wherein said administration is carried out by the oral, nasal, transdermal, pulmonary or parenteral route.

22. A method for identifying compounds for the treatment of disorders where a decreased level of plasma FFA is desired, characterised by screening out compounds that specifically inhibit HSL.
